(19) United States
(12) Patent Application Publication
(10) Pub. No.: US 2018/0294048 A1
(43) Pub. Date: Oct. 11, 2018

(54) PATIENT-CENTRIC PORTAL

(71) Applicant: OneRecord, LLC, New York, NY (US)

(72) Inventors: Jennifer Blumenthal, New York, NY (US); Morgan Knochel, Plano, TX (US); Kathryn Skrocki, Shelburne, VT (US); Jared McCaffree, Snohomish, WA (US); David Landa, Brooklyn, NY (US); Jemma Robinson, Edenthorpe (GB); Sara Thompson, Brooklyn, NY (US); George Steven Blumenthal, New York, NY (US); Wayne J. Singer, Summerville, SC (US); James Tate, Asheville, NC (US)

(21) Appl. No.: 16/010,055

(22) Filed: Jun. 15, 2018

Related U.S. Application Data
(63) Continuation-in-part of application No. 14/624,075, filed on Feb. 17, 2015, now abandoned.
(60) Provisional application No. 61/939,967, filed on Feb. 14, 2014.
(60) Provisional application No. 61/969,414, filed on Mar. 24, 2014.

(51) Int. Cl. G16H 10/60; H04L 29/06

(52) U.S. Cl. CPC G16H 10/60; G06Q 10/1095; H04L 63/0272

(57) ABSTRACT

A system for obtaining substantially all medical information available to a specific patient from a network of endpoints and combining the information to create a comprehensive aggregate patient record. The system allows users to access and download their medical information from numerous institutions. The system combines a user's information into a single aggregate record without requiring the patient to interact with numerous patient portals.

This patent application is a continuation-in-part of U.S. patent application Ser. No. 14/624,075, filed on Feb. 17, 2015 and now pending, which in turn claims priority to U.S. Provisional Patent Application No. 61/939,967, filed on Feb. 14, 2014 and U.S. Provisional Patent Application No. 61/969,414, filed on Mar. 24, 2014, the contents of each of which are incorporated herein by reference in their entireties.

### BACKGROUND OF THE INVENTION

The Field of this invention is secure computerized medical records management. Patients have access to their medical records and health data on a computer, smartphone, tablet, or any other smart device.

Medical records have become computerized. Electronic medical records have many advantages over paper medical records. Electronic medical records aim to reduce the burden of the patient having to remember their medical history, reduce duplicative testing by different doctors, and provide quicker access to the most relevant health data. To facilitate organized record keeping by different institutions, the U.S. Office of the National Coordinator (ONC) has encouraged establishing a uniform architecture for the maintenance of medical records. In response to this encouragement, a markup standard was developed by Health Level 7 (HL7) called the Clinical Document Architecture (CDA). After years of different interpretations of the CDA template, the ONC streamlined commonly used templates to create the Consolidated Clinical Document Architecture (C-CDA). The C-CDA was developed as an XML-based markup standard that specifies the encoding and organization of electronic clinical documents suitable for exchange between health treatment centers and/or patients. The C-CDA provides a base standard with a common architecture, coding, semantic framework, and markup language for the creation of electronic clinical documents. Its structure is object oriented, making use of classes, associations, and inheritance. C-CDA defines building blocks which can be used to contain healthcare data elements which can be captured, stored, accessed, displayed and transmitted electronically for use and reuse in many formats. Sets of C-CDA standardized building blocks can be arranged for different needs. Clinical documents are produced by arranging or constraining the C-CDA elements in different ways.

In 1996, the United States Congress enacted the Health Insurance Portability and Accountability Act (HIPAA), which provides data security and privacy standards for medical information and promoted the establishment of national standards for electronic healthcare transactions. In 2000, the Department of Health and Human Services enacted the HIPAA Privacy Rule. The Privacy Rule generally requires HIPAA covered entities (health plans and most health care providers) to provide individuals, upon request, with access to the protected health information (PHI) about them in one or more “designated record sets” maintained by or for the covered entity. This includes the right to inspect or obtain a copy, or both, of the PHI, as well as to direct the covered entity to transmit a copy to a designated person or entity of the individual's choice. Designated record sets are defined as medical records and billing records about individuals maintained by or for a covered health care provider, enrollment, payment, claims adjudication, and case or medical management record systems maintained by or for a health plan, or other records that are used, in whole or in part, by or for the covered entity to make decisions about individuals. This last category includes records that are used to make decisions about any individuals, whether or not the records have been used to make a decision about the particular individual requesting access.

The United States Department of Health and Human Services has implemented the C-CDA for Meaningful Use Stage 2, which standardizes Electronic Health Records (EHRs). Before EHRs, vast amounts of patient data were collected by clinicians and medical information, such as vitals, orders, prescriptions, lab notes, discharge summaries, etc., which had originally been dictated or recorded by hand. All of these clinical data were stored as paper records (documents) at each point of care. If patient health records were needed to be shared between providers, they usually required manual exchange (e.g., fax, “snail mail”). As a result, the coordination of care between providers was slow and costly and patient outcomes were correspondingly inconsistent. One result was frequent duplicative healthcare services (e.g., labs imaging).

The expected savings to be achieved through computerization of patient records has encouraged government sponsored programs for medical practitioners and for organizations including clinics and hospitals to use computerized records. Financial incentives have been offered for systems that offer patient access to their medical records. Those incentives are increased substantially where it can be demonstrated that the patients are actually making use of the availability of access to those records. ONC: Standards, Implementation Specifications & Certification Criteria (S&CC) 2014 Edition specifies the capabilities and functions that complete EHRs and EHR Modules must perform electronically in order to be certified under the ONC Health IT Certification Program. Meaningful Use (MU2) objectives sets the measurable benchmarks that Eligible Professionals (EPs) and Eligible Hospitals (EHs)/Critical Access Hospitals (CAHs) must meet in adopting and using electronic health record (EHR) technology to qualify for Medicare and Medicaid incentive payments. MU2 sets measurable objectives for EPs or EHs/CAHs to obtain CMS incentives. The MU2 objectives are categorized to reflect Health Outcomes Policy Priorities. These include care coordination and patient engagement, both of which involve using Certified EHR Technology that has the capability of generating a C-CDA. The Care Coordination certification category requires two certification criteria, Transition of Care and Data Portability. The Transition of Care criterion requires that when transitioning a patient to another care setting, the EP or EH/CAH should provide a summary care record. The Data Portability criterion requires that when a patient transitions from one provider or setting to another, a medication reconciliation should be performed, resulting in an export summary.

The Patient Engagement certification category requires two certification criterion, View/Download/Transmit and Clinical Summary. The View/Download/Transmit criterion requires that patients must be able to view and download their own medical information and also be able to transmit that information to a third party. The Clinical Summary criterion requires that a system provide clinical summaries for patients for each office visit. Those summaries are required to include specific fields including patient name, sex, date of birth, race, ethnicity, preferred language, care team member(s), medications, medication allergies, care plan, problems, laboratory test(s), laboratory value(s)/result(s), procedures, smoking status, and vital signs. This government sponsorship and organization has resulted in many diverse implementing programs. As a result of the requirements stated above, providers may offer their patients a portal to access their information. There are no requirements for these portals to be able to communicate with one another. Therefore a patient can have multiple portals that have their information, thus creating digital silos. As such, absent the functionality of the present invention, patients and other authorized users would be required to log into multiple patient portals in a tedious, one-by-one approach with the potential of missing certain information that could interfere with the continuity of care.

Patients typically have medical records residing at several different institutions, often disparate, each of which may have made its own independent determination how to deal with security issues and how to make those records available to patients. Some institutions have their records available through an Application Program Interface (API) which differs from institution to institution. Attempts have been made to collect and organize the records for particular patients by mastering the details of each of those interfaces. What is lacking is a more efficient system for centralizing the records for a particular patient so that they may be obtained by the patient and those whom the patient authorizes to have access.

Also lacking is a system that is patient-centric, i.e., that makes available to a patient, or to a health provider authorized by the patient, access to specific information relevant to that particular patient. In particular, what is lacking is a system that simplifies for the patient the act of getting access to its information that may be located at different institutions, each of which has its own interface for accessing the information. What is also lacking is a system that is self-executing in the sense that the system continually seeks out and pulls in relevant patient data keeping itself up to date rather than relying upon diverse local systems to push the data to the system.

The examples of the art related to this invention and the limitations of that art are illustrative and not exhaustive. Other limitations will be apparent to those skilled in the relevant art upon reading the specification and studying its drawings.

### BRIEF DESCRIPTION OF THE INVENTION

The following embodiments of the invention are descriptive of ways in which the invention may be implemented and are not intended to be limiting in scope. Some of the embodiments are directed to solving the limitations of the problems identified as background; others are directed to different improvements.

The present invention is directed to a method and system for combining a patient's electronic medical records. The present invention concerns computerized medical records management, and in particular, preferably cloud-based software by which electronically stored patient records and other data are automatically aggregated from multiple sources, where the data are related to a single user/patient, allowing a patient (or authorized representative of the patient) to access and share in a secure fashion with other care-givers (such as family members or paid professionals) to whom they grant access, the patient's medical history and related data, where the record is established in a cohesive non-repetitive arrangement. The related data may include information available to the patient by aggregating content from a plurality of sources, such as but not limited to third party applications or databases related to associated medical situations, such as but not limited to medications, medication pricing, healthcare procedure pricing, conditions, vitals, insurance information, fitness information, and genomics information. The system's chief objective is in having a single collection and access point with accessibility that can be sharable in order to facilitate the continuity of care of such patients for prevention, diagnosis, and/or chronic disease management. Attendant benefits of this method and system include:
Patients have near-real-time access to potentially all of their medical information eliminating risks or burdens to the patient of not having all information, and reducing the need for patients to proactively reach out and seek records from multiple providers.
Reduction of the time spent by patients, or time lag related to calling or otherwise contacting health care providers to request and obtain paper copies of their records from one or more providers
Through efficiencies created by the present invention, the likelihood of duplicative, unnecessary or otherwise preventable medical care is greatly reduced resulting in benefits to healthcare insurance providers as well as to patients.
The presentation of a single patient record, collected from a plurality of sources, provides potential insight into conditions and pharmaceutical interactions previously unavailable.
Patients need only remember a single login ID and password to access all of their electronic health information as opposed to multiple logins for patient portals.
Patients can readily provide controlled secure access in part or whole to others, such as to a medical provider new to the patient, when the patient is being seen for the first time or in an emergency situation, as examples.

The system also takes advantage of the implemented incentives passed by the federal government under the HITECH Act, enacted under the ARRA Act of 2009, that incentivize healthcare providers to utilize electronic health records, and in turn provide patients with access to their electronic health records. Healthcare providers benefit under various incentive programs that require certain measures of efficiency enhanced by the system.

The present invention includes a process which queries a network of endpoints containing disparate portions of a patient's complete health record. The present invention uses FHIR (Fast Healthcare Interoperability Resources) and IHE (Integrating the Healthcare Enterprise) transactions to access, retrieve, aggregate, share, and distribute codified health care, financial, insurance, demographic, and data elements in a HIPAA secure environment. The patient is able to view his/her individual health data elements and documents as well as his/her complete aggregate health record. Keeping the information in its original content enables a clear audit trail to be maintained.

The present invention includes or enables a unique application which is a Patient-Centric Portal/process and subsequent functionality where a patient's healthcare and related data securely reside cohesively online. This Portal serves to be the centralized access point for a repository for all of the patient's healthcare related information: demographics, insurance, pharmacies, emergency contact(s), archived historical laboratory, imaging studies, and all inclusive medical histories. Patient data are accumulated and made available by automated and/or manual acquisition from multiple sources (hospitals, clinics, pharmacies, physician offices, insurance entities, and other points of healthcare services). The patient and authorized representatives are able to view individual documents as well as view an aggregate health record, download those documents and records, and securely transmit data to sources of their choosing when needed.

In this application, the term “Health Record” is used to mean a patient's aggregated (or aggregating) health record.

The present invention uses machine learning algorithms to de-duplicate codified health data. For example, a patient may have seen multiple unrelated physicians for the same condition, thereby resulting in disparate entries on disparate recording systems. In the present invention's de-duplication process, data domain matching rules, typically stored in a database of the system of the present invention are used. Once the information has been pulled into the present system and standardized, the Data Domain Matching Rules of the present invention for each data field are fetched and data are compared field by field. The Data Domain Matching Rules are used for determining duplicity, non-duplicity, or disposition of a record. The Data Domain Matching Rules for each section of a C-CDA document are different. For example, the rules for matching medications are different than the rules for matching conditions. Some rules have two comparison levels and others have more than five. The comparisons for a field are then executed in order of importance, which is defined based on the type of field and what section of a C-CDA it is from. After the comparisons have been completed, a confidence level relative to record matching is set. The confidence level for that field is then compared to the rule for that field. If the confidence level is below the lower bound of the rule, then there is a low probability that the data within the field is already contained within the user's Health Record. If the confidence level is higher than the upper bound of the rule, then there is a high probability of similarity to data that is already contained within the user's Health Record. If the confidence level falls between the lower and upper bounds, the data is presented to the user for confirmation. The user then decides if the data is new or if it is already contained within their Health Record. Based on the decision the user makes, the data is either added to the Health Record or discarded. After each field comparison involving user confirmation has been completed, the verified pair is added to the training data as either a distinct pair or a duplicate pair.

The patient has the ability to differentiate or recognize the specific source of each item of the imported data. This centralized data, that the invention has aggregated, is usable to generate patient-specific education and clinical decision alerts. For example, a patient can be alerted when incompatible pharmacological medicines appear in the Health Record. These triggered alerts can be sent automatically to a patient and/or their representatives (e.g., medical providers) on a real-time basis, using e-mail, text, or other digital information services. Information can be printed, downloaded and exported from the Portal. Secure messaging (Direct Protocol compliant) functionality allows messages to be securely transmitted and received. A dashboard centralizes all messaging functionality. A calendar centralizes all calendar information from the satellite portals as well as allowing manual addition. The Portal contains an updatable list of the patient's physicians, pharmacies and hospitals complete with their phone numbers hours of operation and directions to their location.

### BRIEF DESCRIPTION OF THE DRAWINGS

FIG. 1 depicts a Medical Information System with which the portal system operates.

FIG. 2 depicts an embodiment of the Patient-Centric Portal System of the present invention.

FIG. 3 depicts a Patient-Centric Portal embodiment of the present invention.

FIG. 4 depicts the stages of operation for one of the methods of the data aggregation function of the Patient-Centric Portal.

FIG. 5 depicts a home screen of a preferred embodiment of the invention.

FIG. 6 depicts a screen reached by having selected alerts.

FIG. 7 depicts a centralized patient calendar function of a preferred embodiment of the invention.

FIG. 8 depicts an insurance coverage function of a preferred embodiment of the invention.

FIG. 9 depicts a secure communication function of a preferred embodiment of the invention.

FIG. 10 depicts a graphic depiction of trends in the data of a preferred embodiment of the invention.

FIG. 11 depicts linking to patient's problems, medication and/or health requirements of a preferred embodiment of the invention.

FIG. 12 depicts graphic information on income going to medical health of a preferred embodiment of the invention.

FIG. 13 shows a screen where the user can initiate a Super Query.

FIG. 14 shows the data flow for the User Initiated Super Query which involves searching for new records across multiple responding gateways.

FIG. 15 describes a machine learning rules based process for identifying duplicate data with the user in the loop.

FIG. 16 shows the ability for users to prepare for doctors appointments by filling in a questionnaire and adding notes and questions that they want to discuss with their doctor.

### DETAILED DESCRIPTION OF EMBODIMENTS

The present invention is directed to a method and system for aggregating a patient's medical data records across a plurality of sources, such as but not limited to physicians, hospitals, pharmacies, and the patient. In aggregating the records, duplicate and/or redundant information is removed, thereby providing a concise view of the patient's history and conditions. Interactions between symptoms and mediations, for example, can be identified readily and potentially automatedly. As the patient receives further treatment or further visits a physician, as examples, the record is updated and, potentially in real time, contraindications can be identified, even when not mentioned by the patient or identified by the physician. Such an approach has the potential to aid physicians in diagnosis and treatment and has potential to save lives.

Further, such analyses could be, at least in part, automated, to trigger avoidance of certain medications or to alert a patient, as examples.

The core portion of the system of the present invention includes:
A secure patient Portal, preferably accessible through a Graphical User Interface (GUI) for arranging (1) for an aggregated Health Record, (2) for viewing attributes of the record, (3) for providing views into educational materials and associated medical conditions, such as providing alerts, (4) for assessing and eliminating the possibility of duplicity in records, and (5) for providing access to the patient's Health Record to others, such as a physician.
A database for storing Health Records and rules associated with the arrangement and aggregation of Health Records.
A processor array (or a single processor) for communicating with third party facilities with portions of a patient's Health Record and for extracting those portions, as well as for processing rules.
The processor array further communicating with the patient, such as through the Portal, as well as outside sources, for determining and identifying educational and alert situations.
A de-duplication engine for recognizing the likelihood of duplicated records, communicating with the database accordingly, and for interacting with a patient when the rules result in non-automated deterministic results.
The processor array also updating the stored rules in the database based on patient-identified results.

The present invention provides an improved method and system for a patient and his/her authorized representatives to access and manage health care based on the patient's complete electronic medical record and health information. A user, or a patient, and/or their authorized representatives, can access the patient's data after authenticating their identity. Medical records and health information, in the form of C-CDAs, other codified documents, and non-codified documents such as PDFs and images, are pulled into the patients' account as individual documents. Each individual document is then compared to existing information in the patient's growing medical record in order to de-duplicate information and leave the patient with a single aggregate medical record. The sources of this medical information include but are not limited to health systems, hospitals, private practices, individual providers, health information exchanges, insurance companies, genetic testing companies, genetics companies, and health industry data sharing consortiums.

Data Inclusion:

Data for the system may be acquired from queried endpoints or by file uploaded by the patient using the Portal (or comparable means), either directly or indirectly in the form of XML documents (CDAs and C-CDAs), DICOM, PDF, JPEG, PNG, and/or GIF files. Data may also be typed into the system by the patient, or acquired via connected devices such as biometric or fitness devices.

The methods for finding data for a specific user include sending queries to the network using a user's demographic information, sending a user's demographic information to a Record Locating Service, and/or sending a Medical Record Number that has previously been identified as being associated with the user. Documents and data found are then retrieved by the system. Before the documents and data are deposited into a user's account, they are checked again to make sure they match the demographic information in the user's account. If they match, then the documents undergo a hash check to see if the specific document already exists within a user's account. If the hash check passes, then the document is deposited into the user's account. If the hash check fails, then the document already exists within the user's account and therefore is not brought in again. Preforming these detailed checks ensures that the PHI belongs to the user requesting information.

The system of the present invention provides on demand patient specific educational resources based on system patient data. As patient data populates the system of the present invention, the system can also populate educational resources, such as links to articles and information for the user related to the particular item. This information may be displayed within a frame on a system display (or Portal), allowing the patient to stay on the patient-centric Portal without having to exit the system (website), or through launching another browser window or tab with a third party website. Relevant links to some of the most well-known healthcare information websites are also provided (e.g., WebMD). Additionally, there is a search function in the patient-centric Portal that allows a patient to enter a search that will display results within a window on the Portal. A customization feature will allow the patient to save links to their own favorite resources.

Types of Access

The portal system provides patient access, authorized representation access (with read only or read/write capabilities), and In Case of Emergency (ICE) access. In each instance, access to specific data is controlled by tags indicating who has access to and then who accesses the patient's data. In that manner, the patient or a health provider can determine how widely the patient's information is shared.

In some circumstances, it may be beneficial to concurrently display data regarding a plurality of patients. Such a display would be (1) with full patient authorization and/or (2) with patient anonymity, such as in attempt to determine the presence of an epidemic. The information relating to a plurality of patients may also be displayed in a portal view for a medical provider.

The system also allows Friend and Family Access, for example by providing a dashboard view for multiple patients, and Case Manger Access providing a dashboard view of numerous patients with ability to apply specific continuity of care protocols based on one or more data elements (problem list, medication list, medication allergy list, lab test results, and third party payers).

Functionality

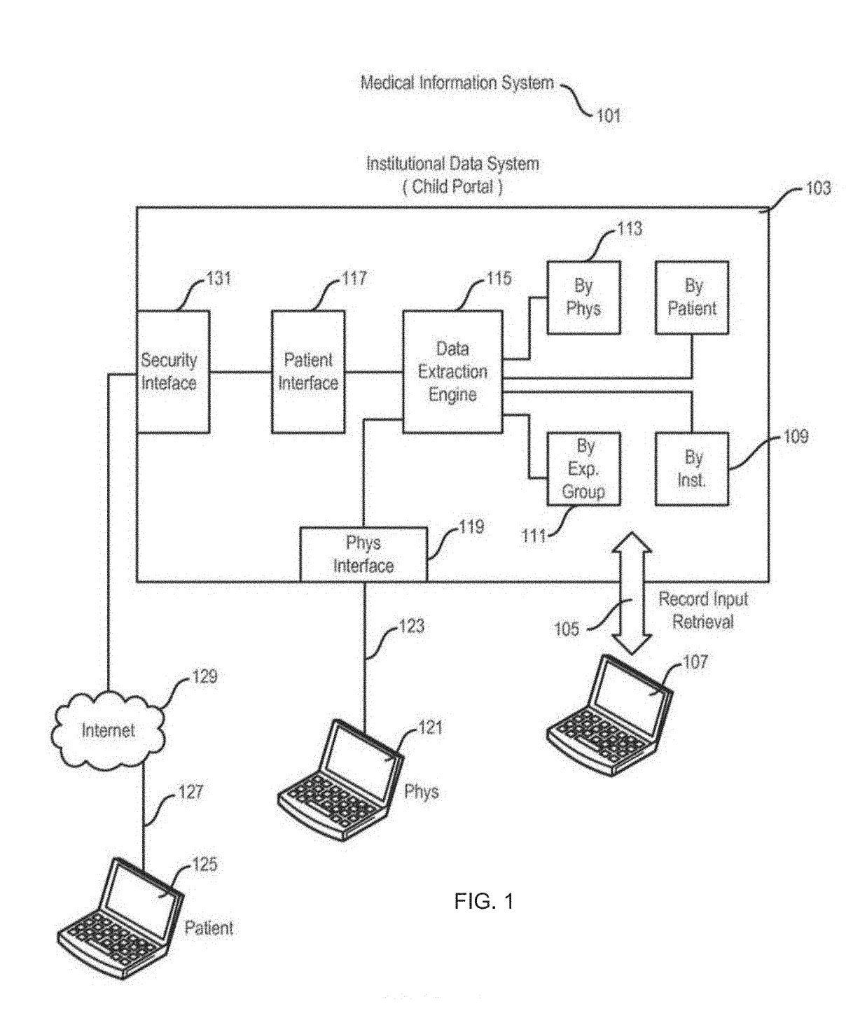

FIG. 1 depicts a Medical Information System with which the portal system operates comprising one or more Institutional Data Systems 103, also termed a Satellite Portal. Each Institutional Data System 103 is designed to receive medical information over a network 105 from physicians, medical practitioners or laboratories from Record Input and Retrieval computer equipment 107. The network 105 may be a wired LAN or WLAN or with proper security may be established by remote communication. Acceptable communication protocols may include WCDMA or GSM communication systems, and the computer equipment 107 may be a terminal or tablet, smart phone or a PDA.

The Institutional Data System 103 has one or more databases of medical information that may be centralized in a singular database with multiple fields or divided into separate databases according to how the information is received or expected to be accessed. For example, the database of information may be separately maintained 109 according to the institution or department of an institution that created all or a portion of a particular data record. The information may be separately maintained 111 by the expert group, for example a laboratory, that created all or a portion of a particular data record. Such a laboratory could be a radiology group or a screening laboratory. The information may be separately maintained 113 for each physician or practitioner that is permitted to create records in the Institutional Data System 103.

Within the Institutional Data System 103 there is a Data Extraction Engine 115 which can respond to requests for particular types of records, by extracting responsive records from the informational databases 103, 109, 111, 113. The Data Extraction Engine 115 is provided with an interface to those requesting information. The interface may be comprised of separate interfaces generally termed a Patient Interface 117 and a Physician Interface 119, although the two interfaces may be combined into a single structure able to distinguish whether the information being sought through the interface is coming from a patient or from another authorized requesting source. Physicians' computers 121 may access information over the Physicians Interface 119 over a network 123, which may be any of the types of network previously mentioned for inputting information. The computer devices 107 121 may in fact be the same device.

The Patient 125 is accessing the information in a Patient Portal via a link 127 to the Internet 129.

Patients using computer devices 125 may seek access to information from the Institutional Data System 103 over a patent access network 125 that may include an Internet 129 portion. The patient access network may have to pass a security interface 131 before gaining access to the Patient Interface 117. In this system, there may be data that is entered and available to physicians but not yet released to patients for them to view or download. Such information may be laboratory results that require analysis and explanation to the patient before being made available to the patient. In this manner, physicians may provide the patient with a prognosis before releasing data that could be upsetting to an uninformed patient.

When the patient's health data includes medications, procedures and other relevant items pertaining to healthcare provider visits, the system will allow the patient access, either through data collected by the System or data gathered from third-party databases, to see the pricing and availability of the medications, procedures, diagnostic images, lab tests and other events for which a patient would incur a cost. A map will display to the patient where they can purchase the foregoing cost-items near their location, or within a designated geographic area of their choice. The cost data will be presented both for retail and generic versions of medications. In each case, the system will calculate the net cost to the patient after applying their particular insurance policy's reimbursement or coverage of such cost, and display that amount. This will enable unprecedented price transparency to patients, a long-stated goal of many healthcare policy experts. Insurance data may be polled from a secure SOAP or MIME IP address supported by the HETS.

Insurance information will also be presented in a manner to allow the patient to track relevant cost information including their deductibles, showing how much the total individual and family deductibles are for their policy, and how much has been used in each category as of the time the user reviews the data. Additionally, they will see all items (medications, procedures, DME) that require prior authorization to avoid unexpected out-of-pocket costs.

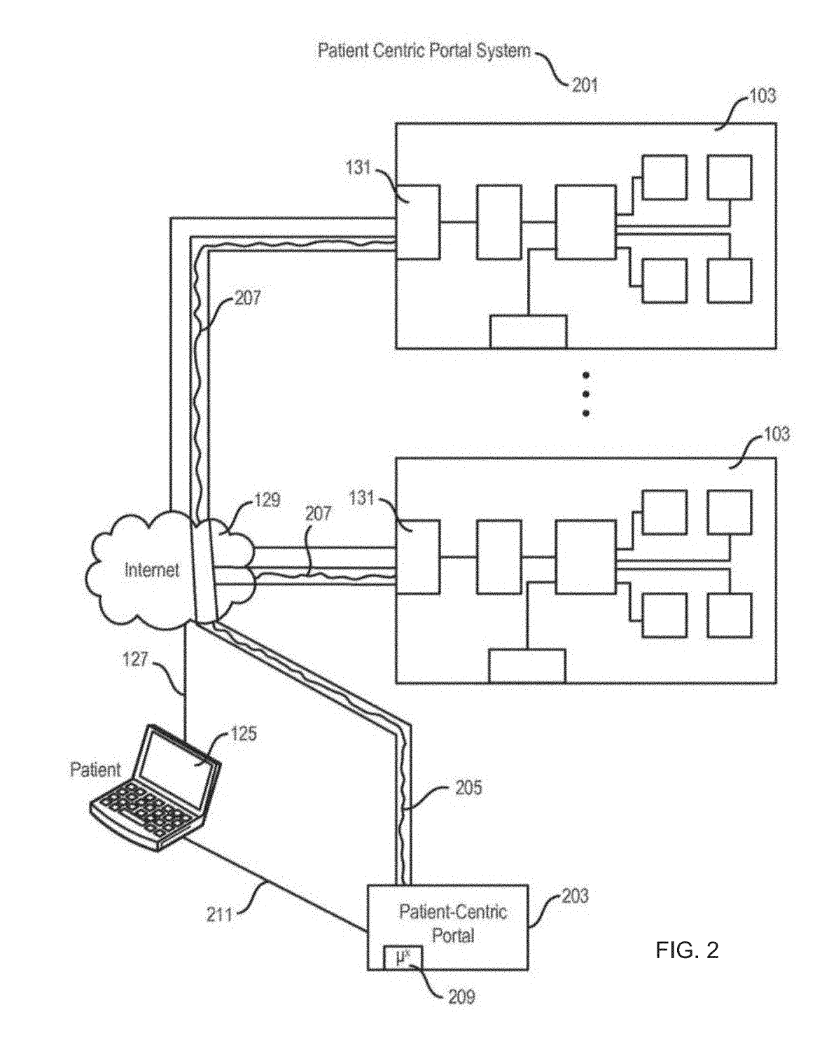

FIG. 2 depicts an embodiment of the Patient-Centric Portal System of the present invention. The Patient-Centric Portal 203 is interfacing with multiple Patient Portals 103. There can be any number of Patient Portals 103 that can interface with the Patient Centric Portal 203.

The Patient Centric Portal 203 maintains access to each of the interfaces 131 accessible to patients, via tunnels 205, 207 over the Internet. The tunnels 205, 207 permit secure communication between the Patient-Centric Portal 203 and the Institutional Data Systems 103, with the ability to access the same information as may be accessed on the patient's computer 125. The tunnels 205, 207 are maintained by encrypting and subsequently decrypting the information passing to and from the Patient-Centric Portal 203 and the Satellite Portals 103. The encrypting/decrypting function is established by a codec maintained by a processor 209, which is a component of the Patient-Centric Portal 203. Via a portion 205 of the tunnel, the patient and the Patient Centric Portal 203 may communicate directly to exchange information that the Patient Centric Portal has derived from the Satellite Portals. In this manner, the Patient-Centric Portal may act as an agent for the patient and provide in a centralized format the information derived from a plurality of Satellite Portals 103. In addition, the Patient's computer 125 may communicate directly with the Patient-Centric Portal 203 over a network connection 211 established for that purpose. It is understood that as used here the concept of tunneling is meant in a broad sense that may include establishment of a virtual private network to establish the secured connections described herein.

Data may be accessed and retrieved from portals 103 by the Patient-Centric Portal 203 on demand and by automatic time configurable polling. The polling is achieved by the various satellite portals having software to recognize the patient-centric portal and permitting it entry into the internal database residing on the portal. Once recognized, the patient-centric portal is enabled to identify information that it is permitted to copy onto the patient-centric portal. In this manner the patient is not required to interact directly with any API that confronts others seeking information from the satellite portal's internal database.

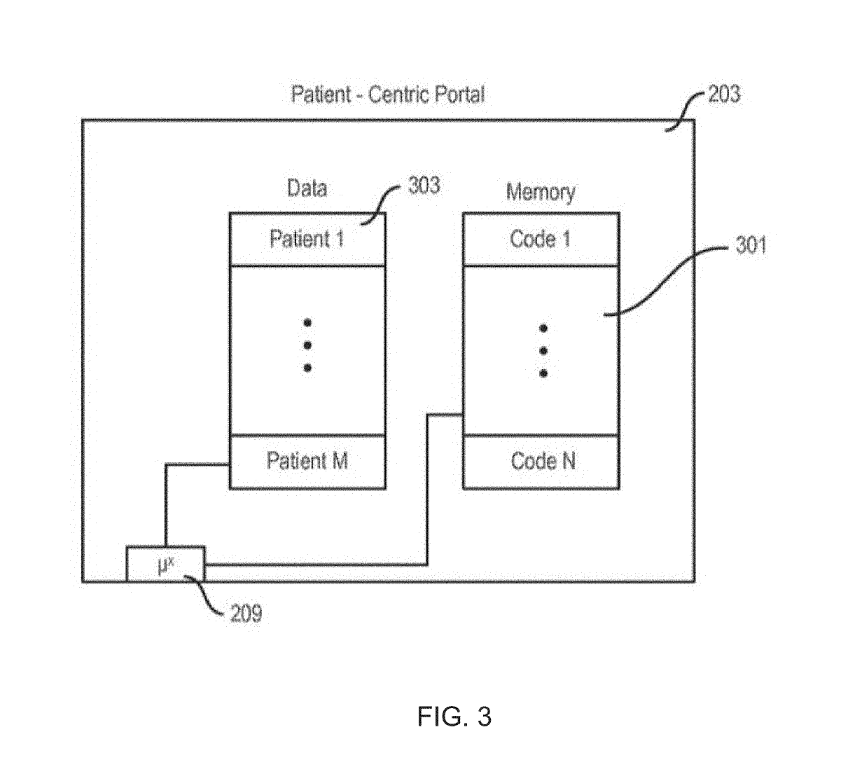

FIG. 3 depicts a Patient-Centric Portal embodiment of the present invention. There can be any number of patients 303 in the system.

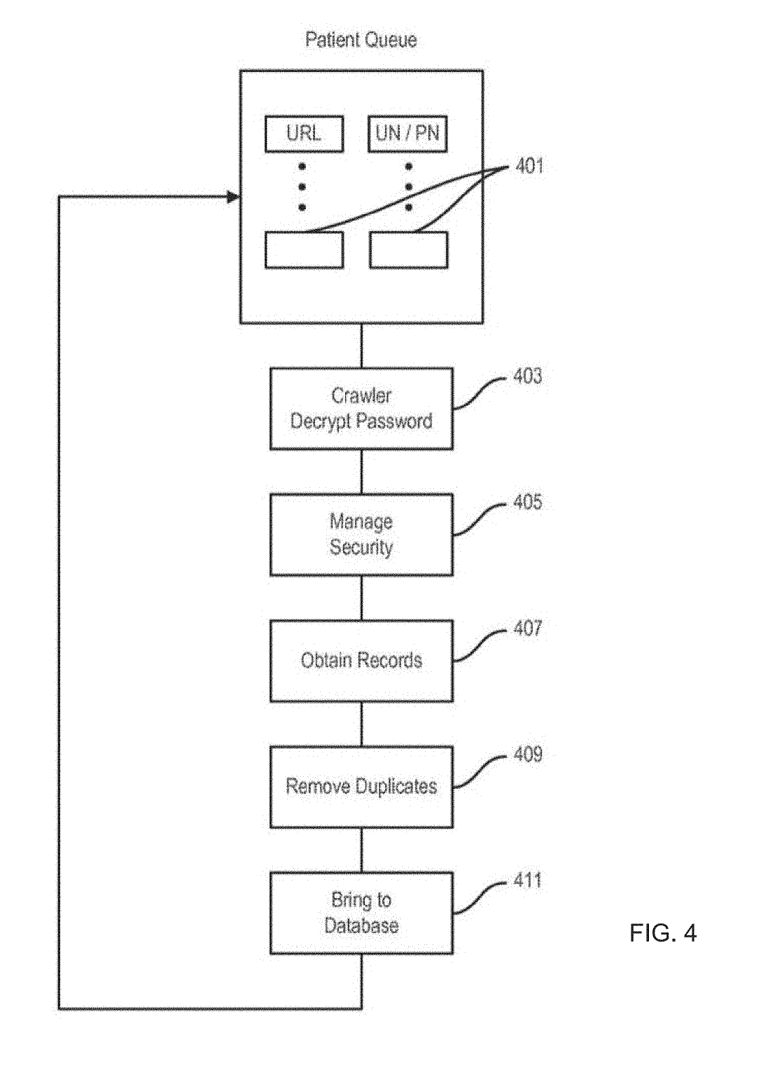

FIG. 4 depicts the stages of operation for one of the methods of data access and aggregation of the Patient-Centric Portal. The system comprises a patient queue in which is contained the URL for each Institutional Data System. The operation begins by selecting a next 401 URL and the associated patient User Name and Password in the queue. The crawler software causes 403 the system to access the next satellite portal.

Where necessary the password information is decrypted. The codec appropriate to the satellite portal is selected by the crawler and security at the satellite portal is provided with authenticating information. In some instances, the satellite portal is guarded by an interface intended to defeat unauthorized web-crawlers by requiring the reduction to text of images that are hard to discern. By a combination of software adapted to reduce images to text, the security is managed 405. The records relevant to the particular patient and made available for access by the patient are obtained 407, duplicates are removed 409 and the patient data is brought 411 into the Patient-Centric Portal's database.

The Patient-Centric Portal is adapted to perform multiple functions with the data obtained from the satellite portals in addition to merely making the data available to the patient in a centralized location. Those functions comprise additional features of preferred embodiments of the invention.

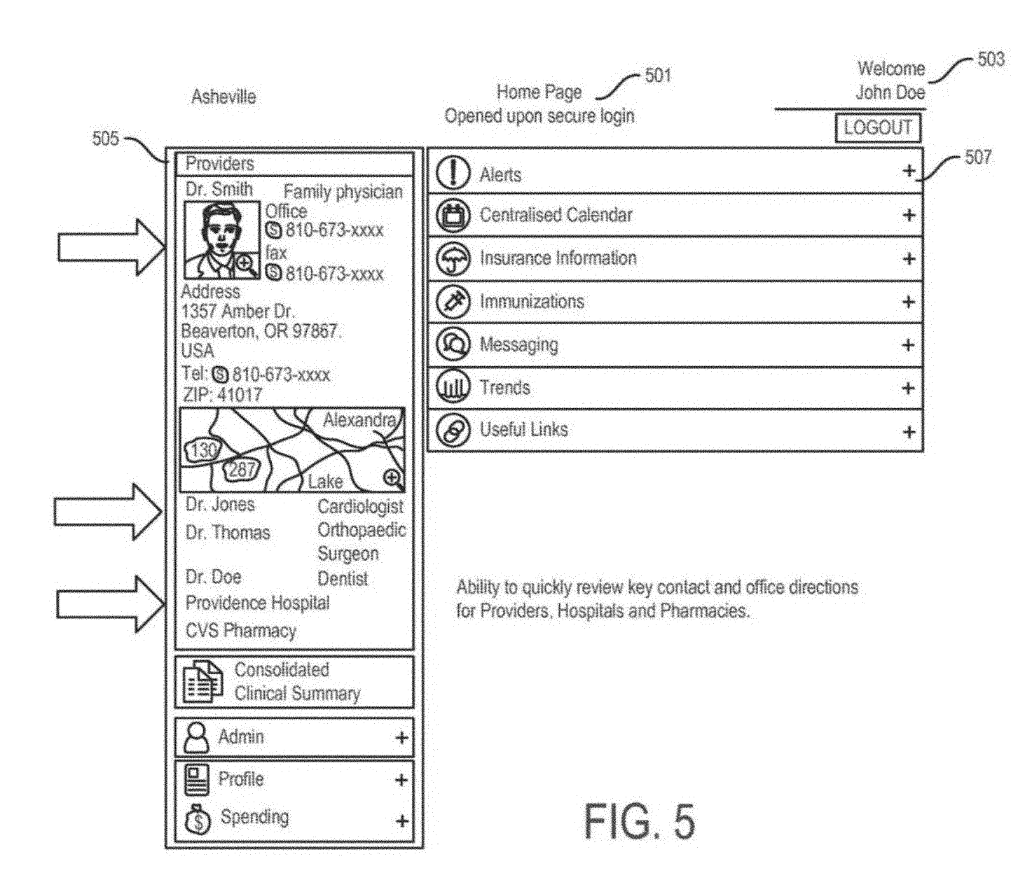

FIG. 5 depicts a home screen 501 displayed upon patient access via secured login to the Patient-Centric Portal System. The screen identifies the patient 503 and provides access to information on the various information providers 505 and provides office direction for providers, hospitals and pharmacies. In addition there are links for different major functions 507 of the system. These include Alerts, a Centralized Calendar, Insurance Information, Immunizations, Trends and other Links.

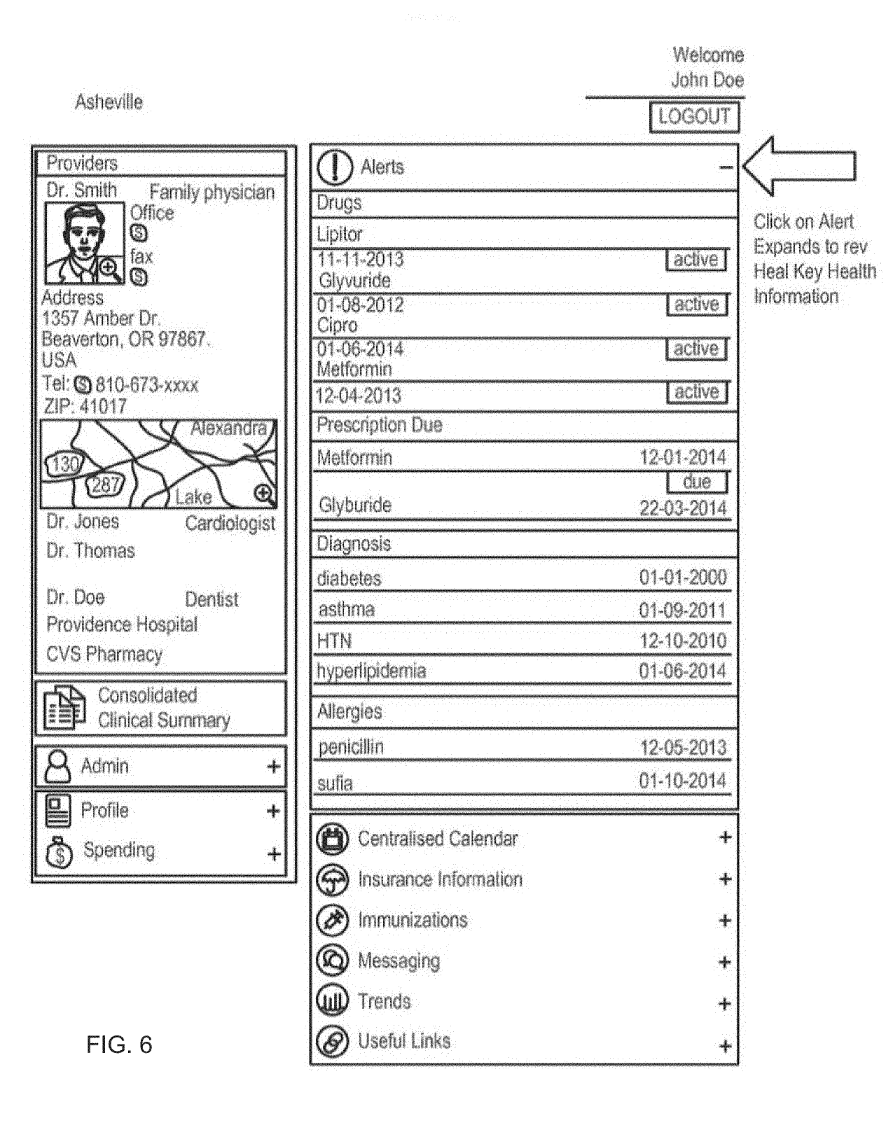

FIG. 6 depicts a screen reached by having selected alerts, to reveal key health information including prescriptions due, diagnoses and allergies.

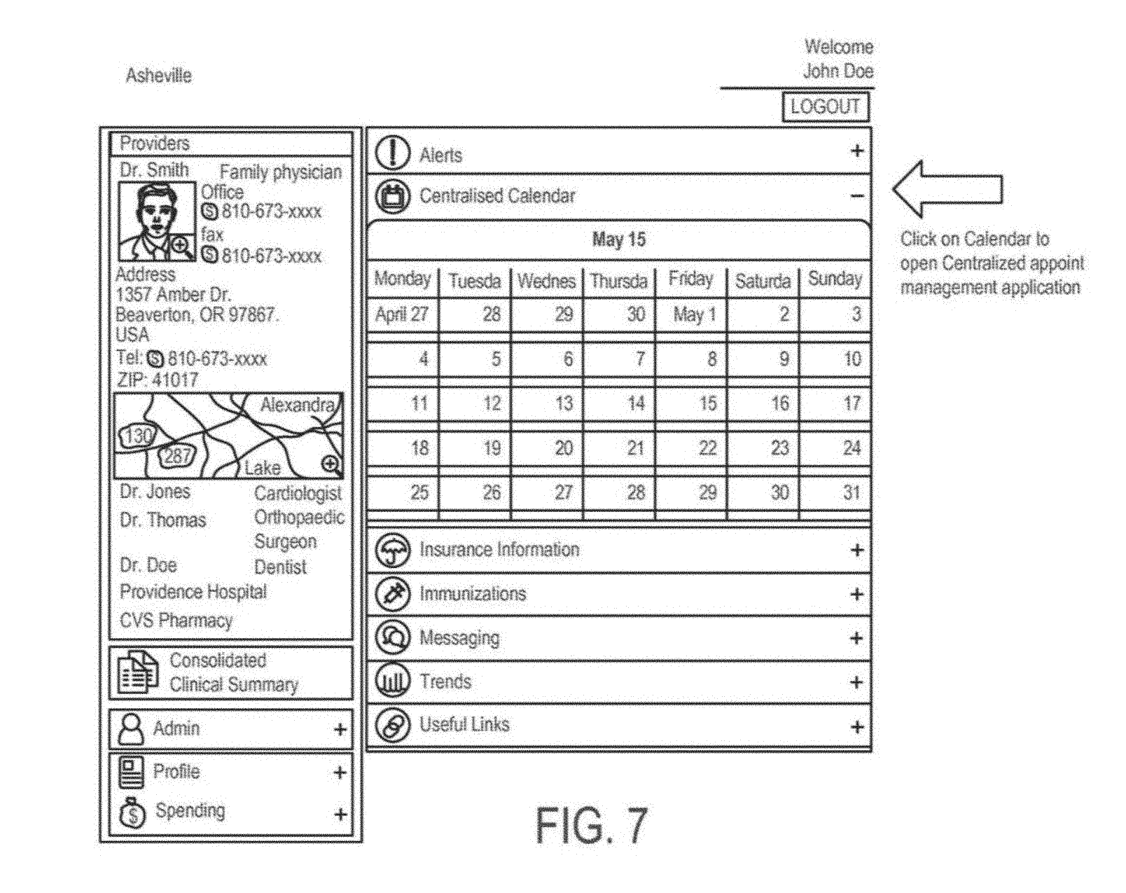

As depicted in FIG. 7, the patient-centric portal system has the ability to support a centralized patient calendar (to maintain appointment lists, medication refills and transportation requirements). The calendar will centralize all calendar information from the satellite portals as well as allow manual addition. The calendar will provide the incorporation of configurable reminders that can be sent to the patient, and/or their authorized representative.

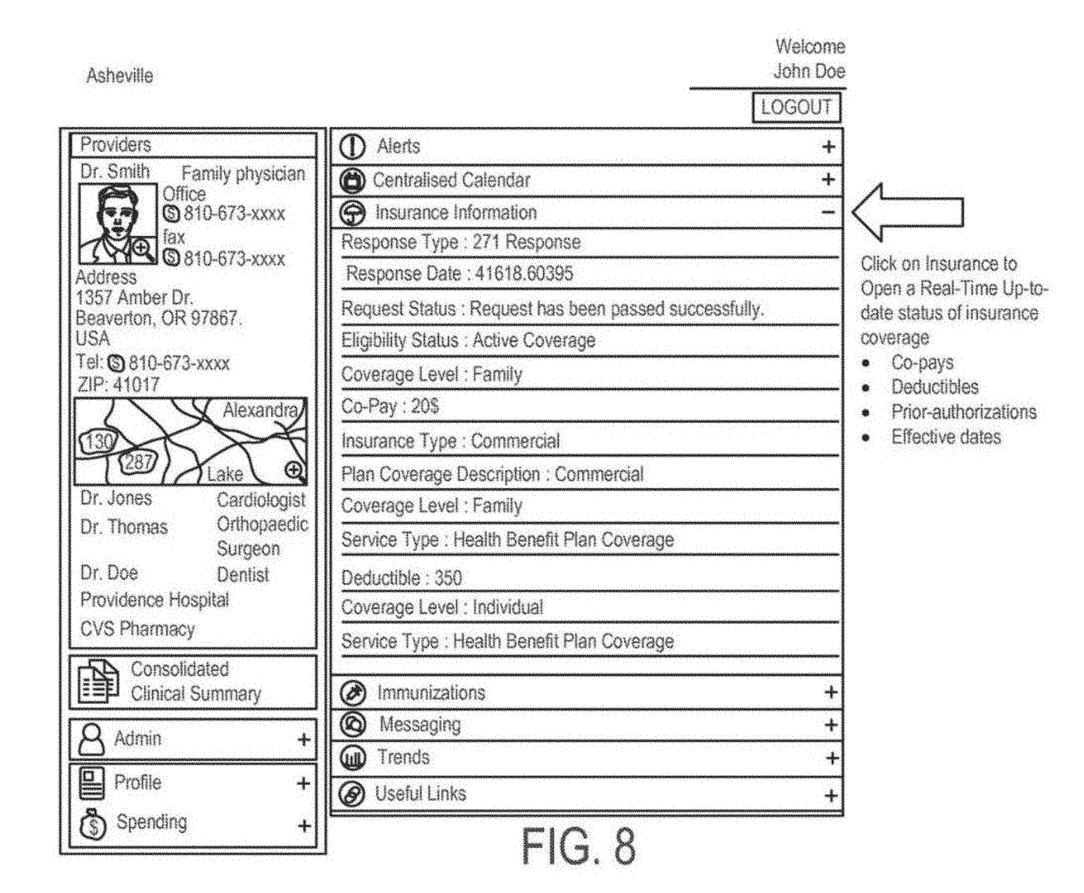

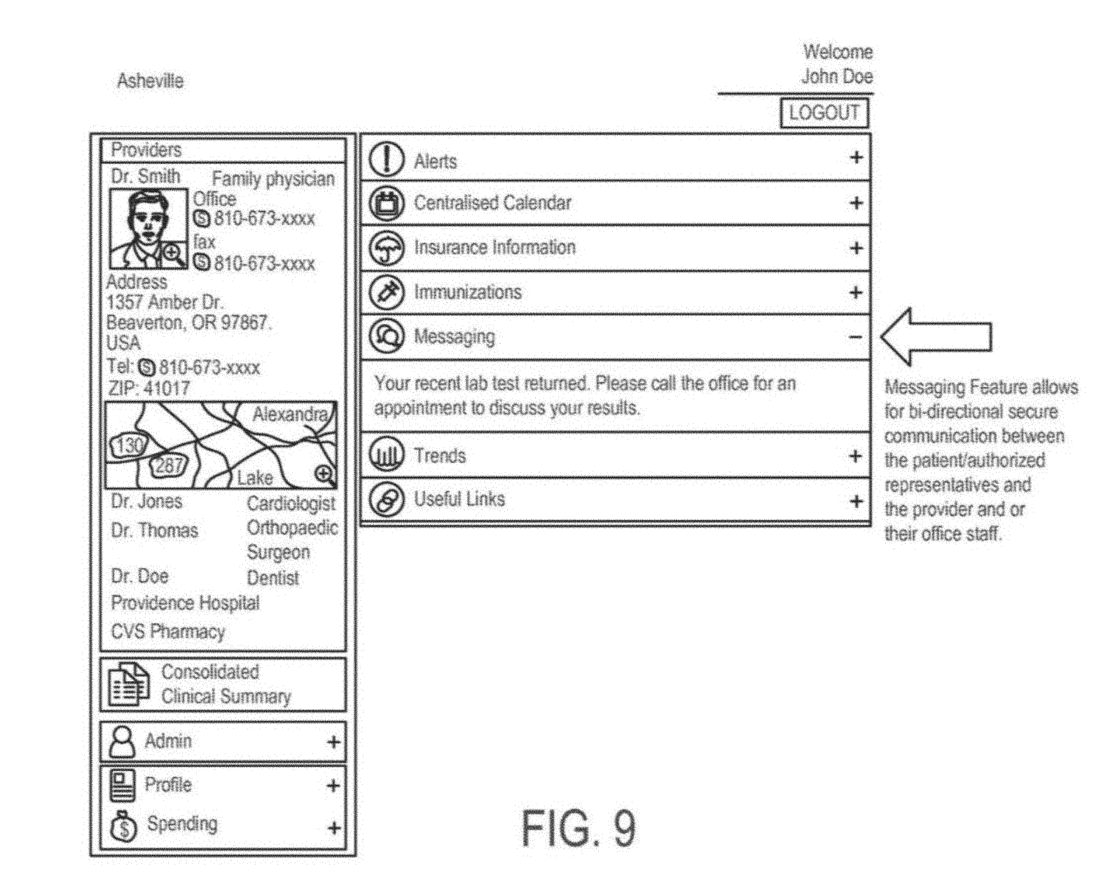

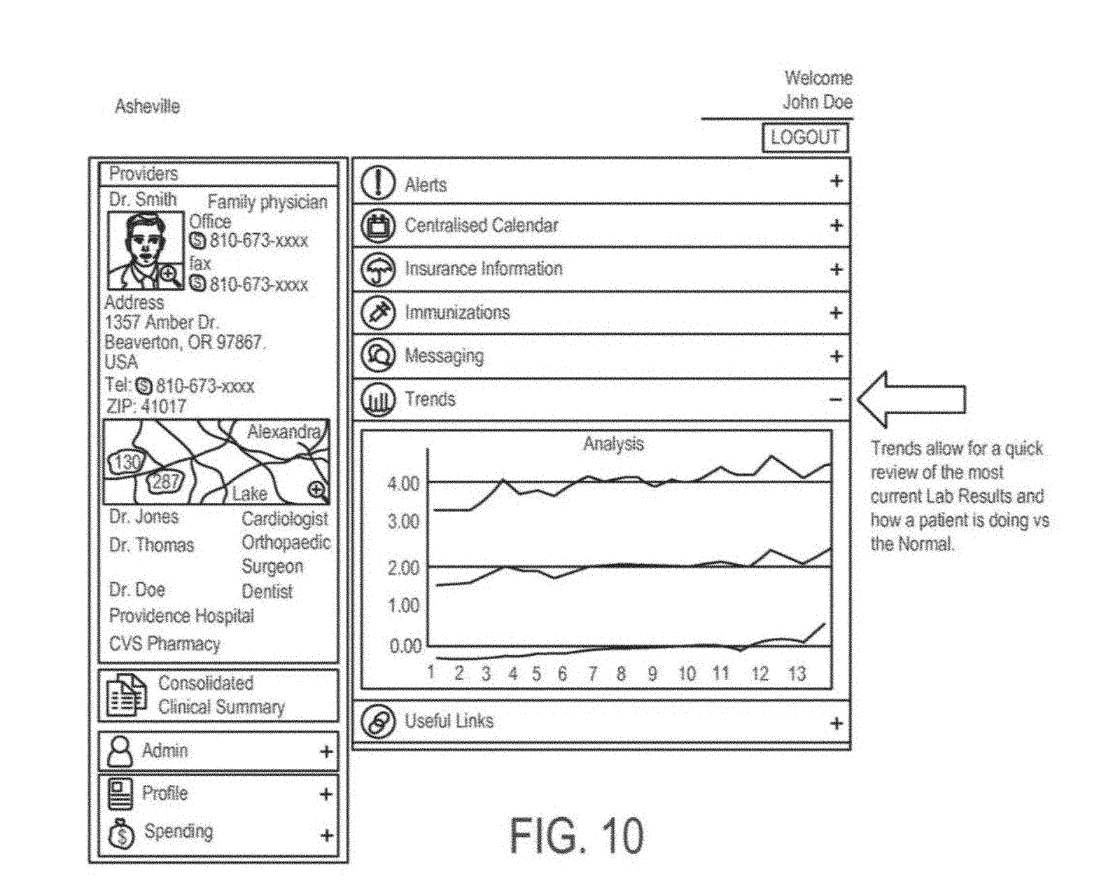

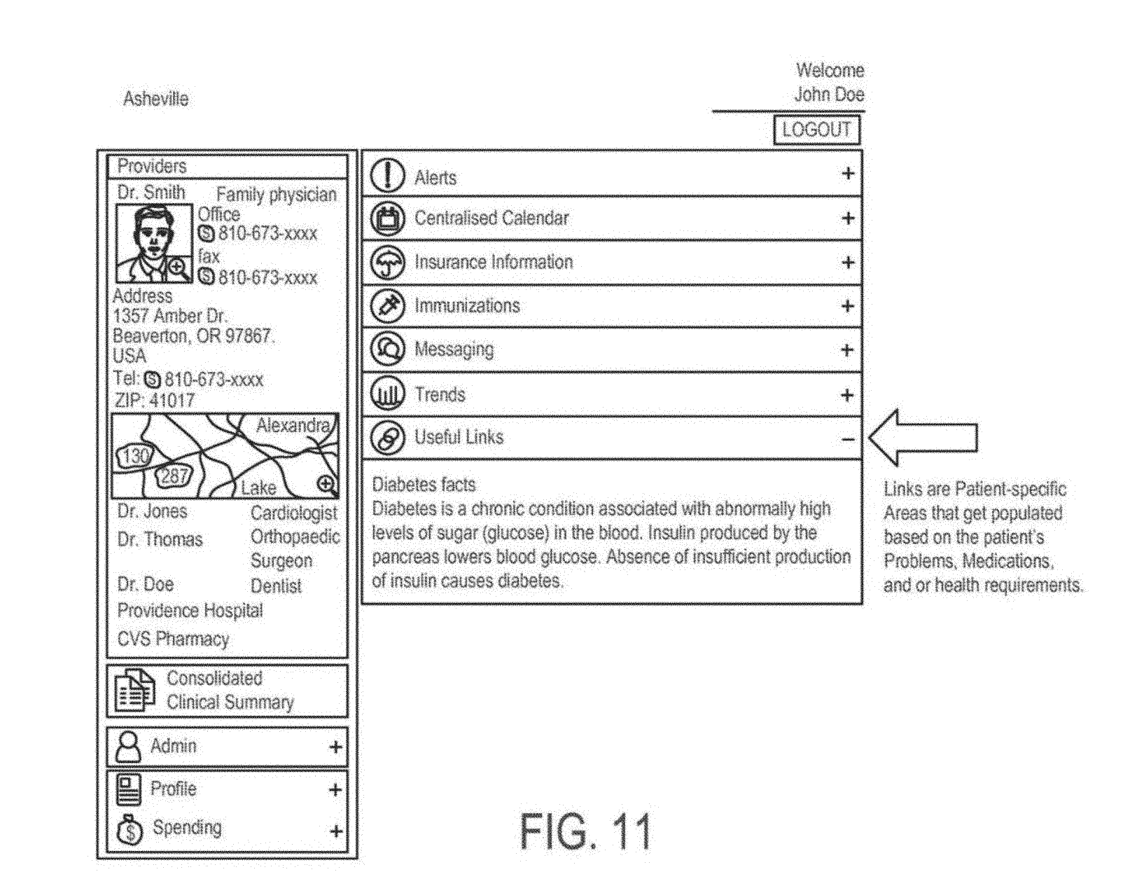

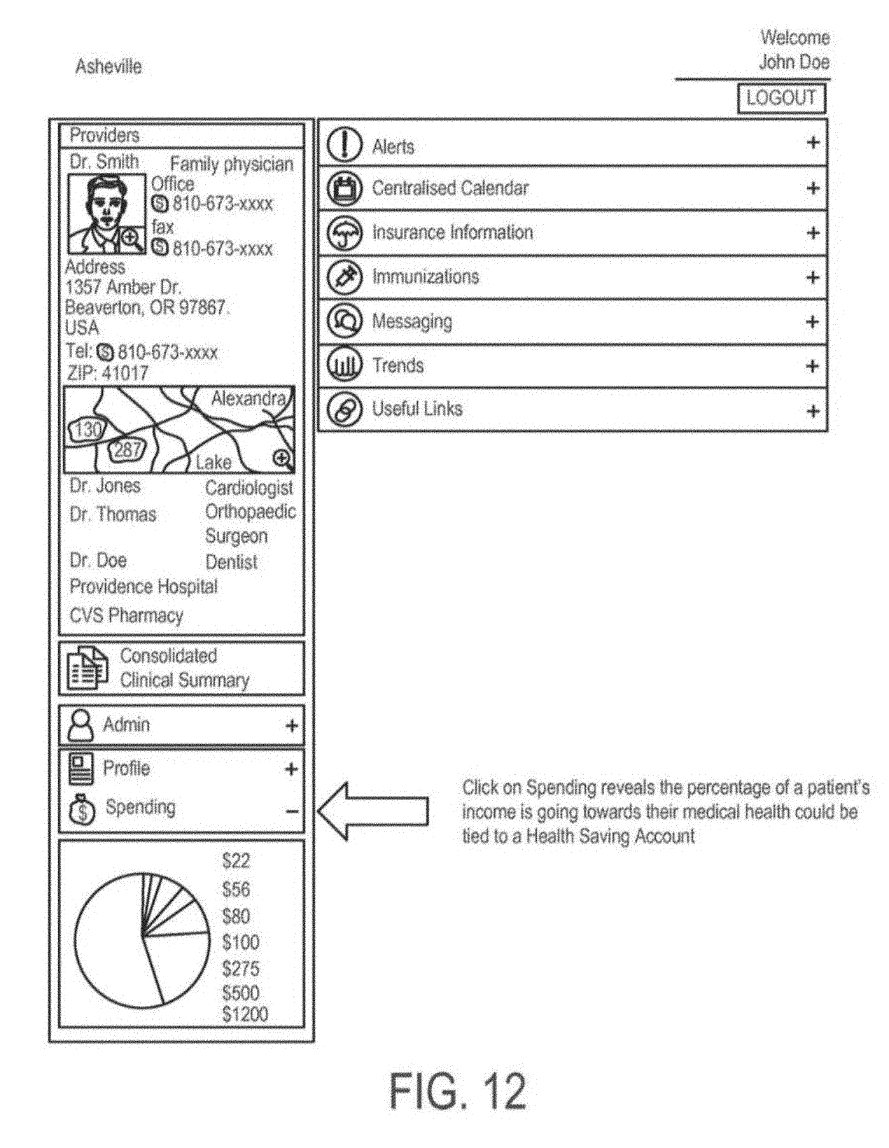

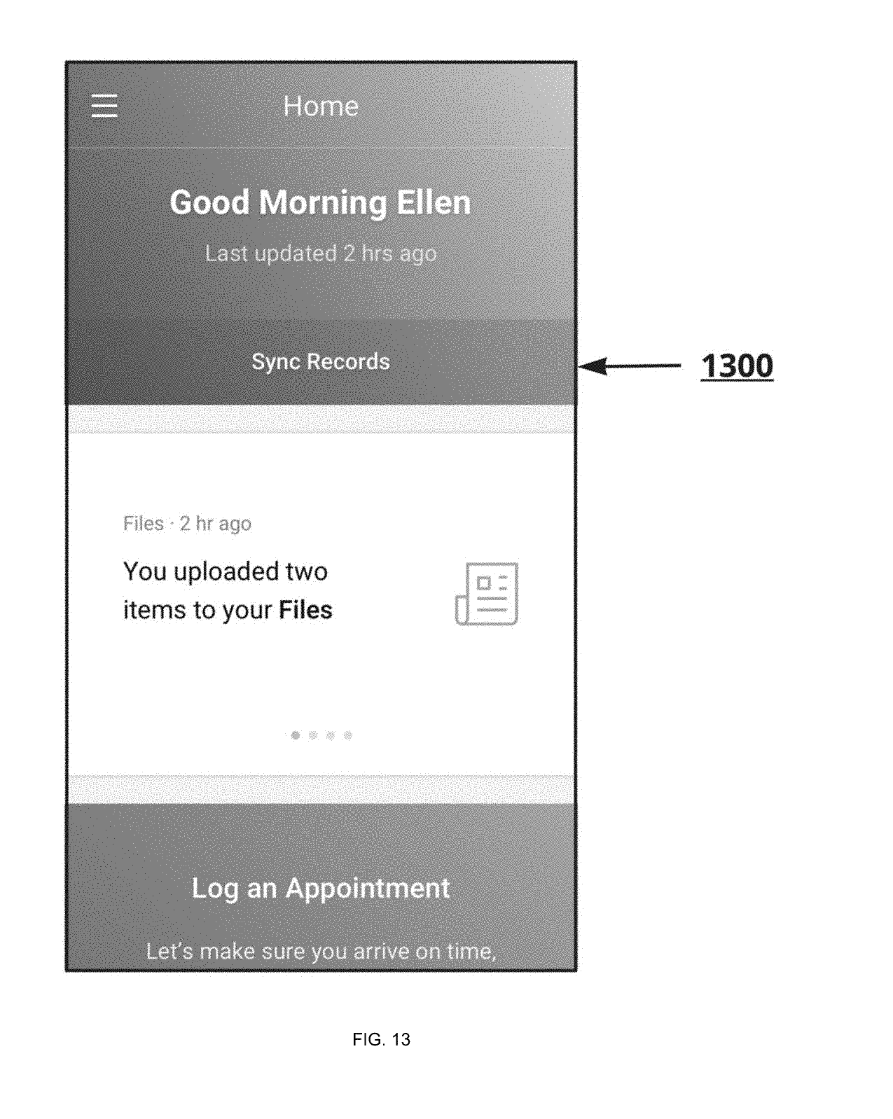

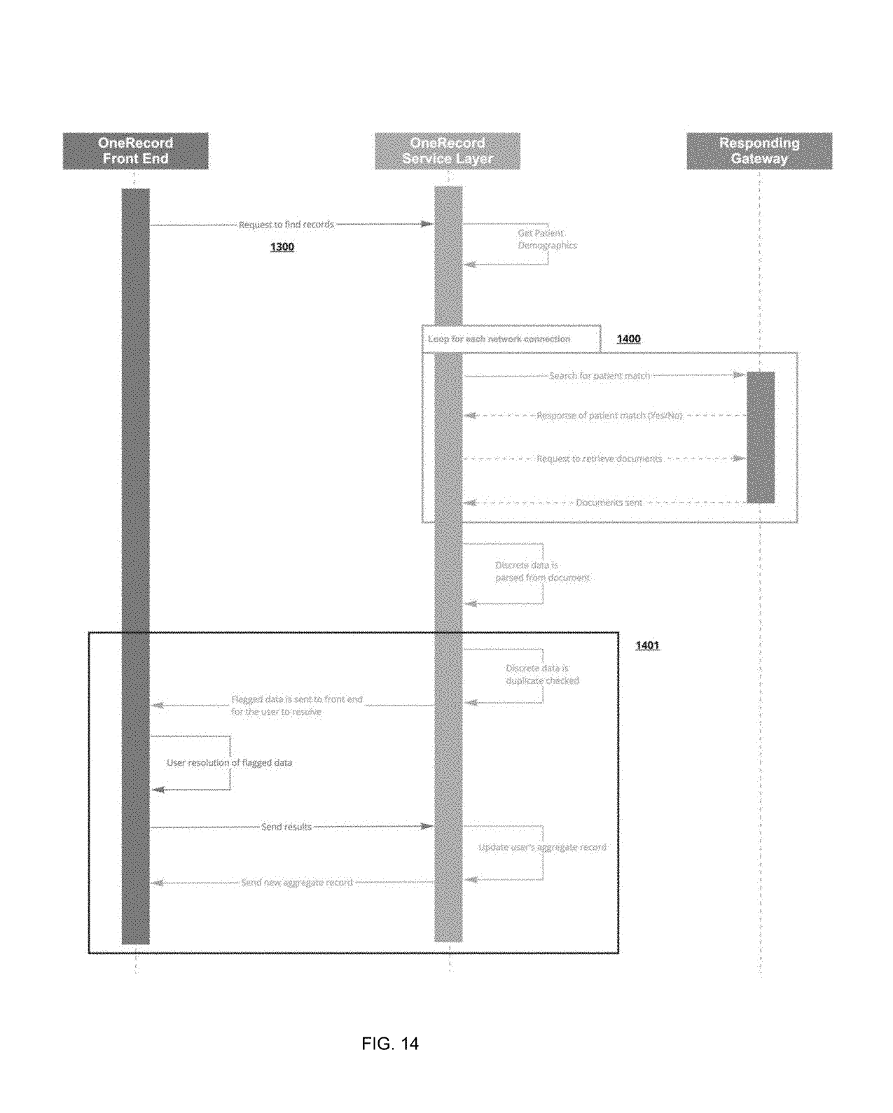

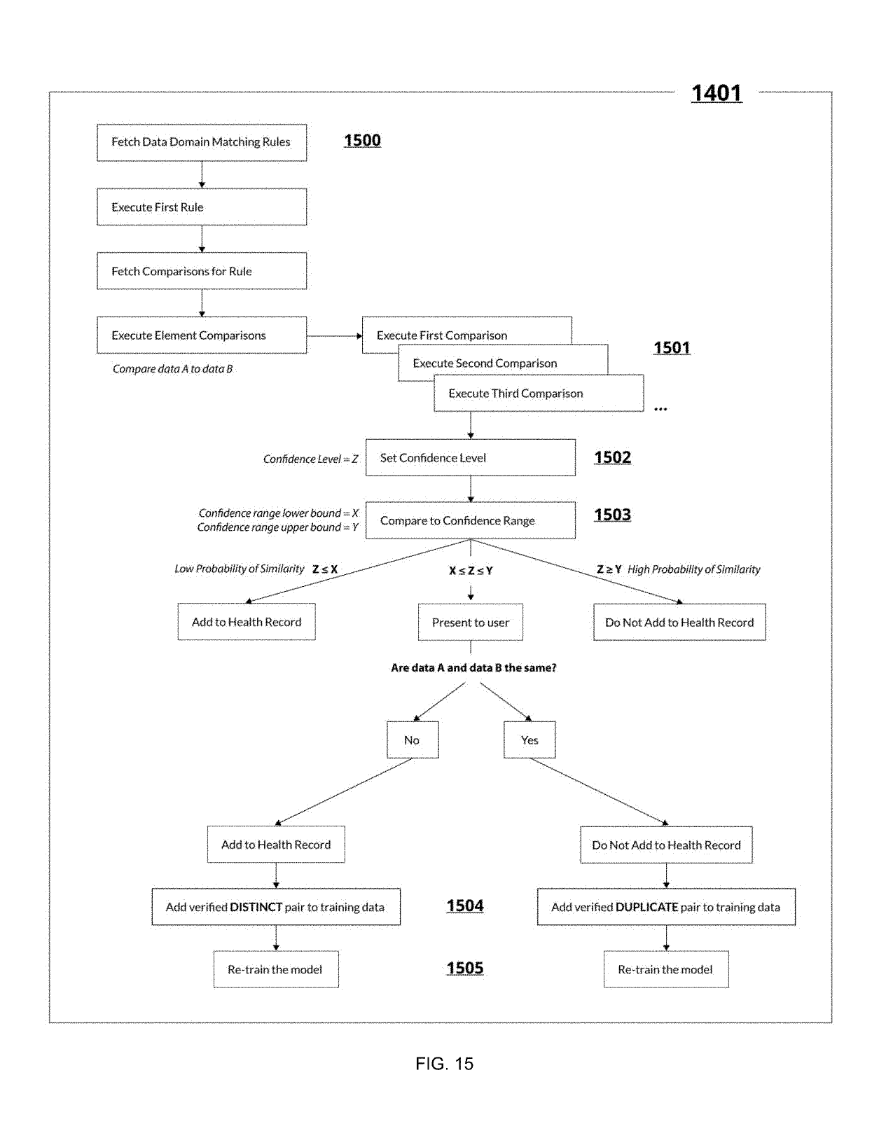

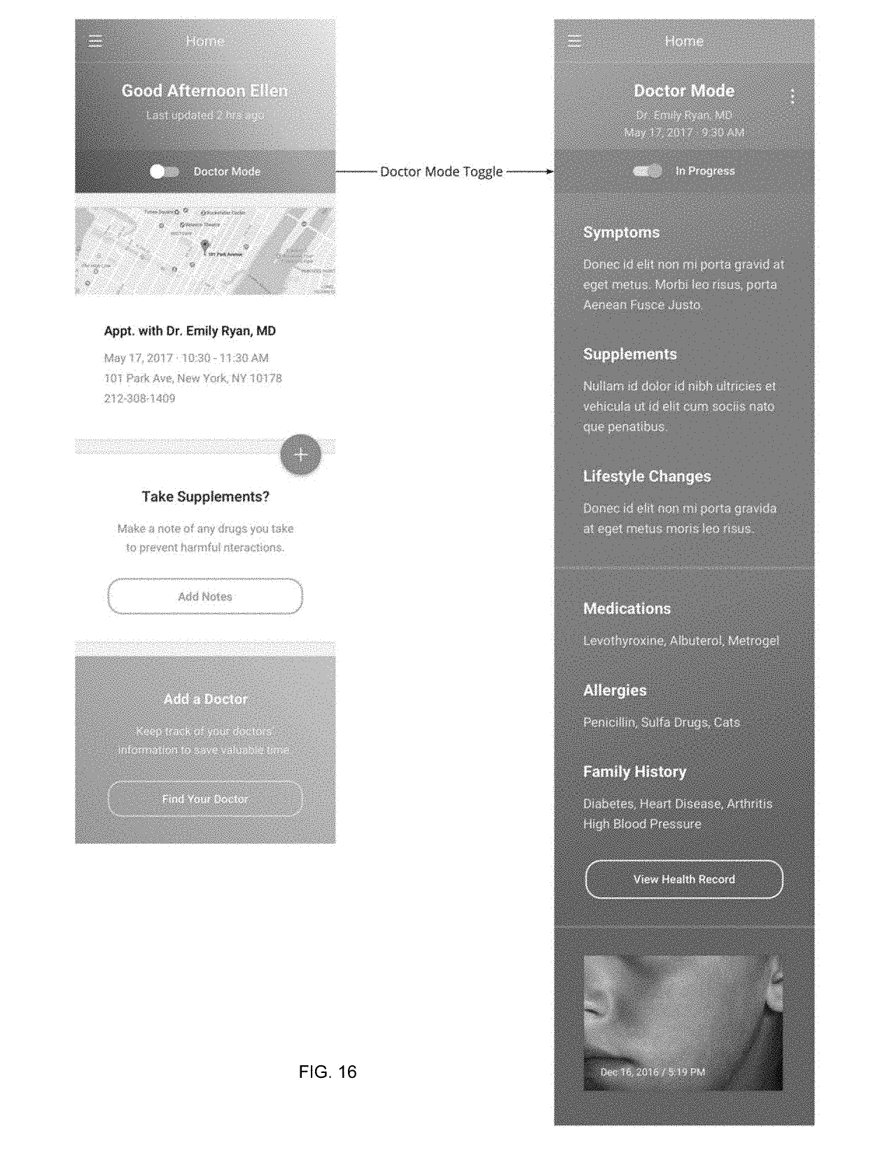

As depicted in FIG. 8, the system provides real-time up-to-date status of insurance coverage, including co-pays, deductible, prior-authorizations and effective dates. FIG. 9 provides a screen from which the system allows bi-direction secure communication between the patient/authorized representatives and a provider and/or their office staff. FIG. 10 provides a screen with graphic depiction of trends for the quick review of the most current lab results and a comparison with normal values. FIG. 11 depicts a screen that provides links to areas that get populated based on the patient's problems, medications, and/or health requirements. FIG. 12 provides a screen with graphic information on the percentage of patient income going towards their medical health and could be tied to a Health Savings Account. FIG. 13 shows the screen where the user can initiate a Super Query. A User Initiated Super Query is where a user retrieves their disparate health records from multiple systems. FIG. 14 shows the data flow for the User Initiated Super Query which involves searching for new records across multiple responding gateways. A user initiates the process 1300 to find and retrieve records. Finding records 1400 involves searching for a patient within a Responding Gateway and if there is a match for a patient, retrieving the available records for a patient from the responding gateway. Documents are then parsed into discrete data. The de-duplication process begins 1401 where data is checked against the rules matching database, flagged data is then presented to the user to resolve, then the user's resolutions are sent back to the service layer to be re-incorporated into the de-duplication algorithm. The user is then presented with their aggregate record with the new records incorporated. FIG. 15 describes a machine learning rules based process for identifying duplicate data with the user in the loop. After the data has been discretely parsed, each data domain and element goes through this process 1401. The rules for the data domain are fetched 1500 from a database. The rules for the data element are then fetched and the comparison rules are applied 1501 to the data element. A confidence level is set 1502 based off of the results of the comparisons. The confidence level is then compared to the confidence range for that data element 1503. If the element falls within the confidence range, then it is presented to the user to decide if it is distinct or duplicate data. The user's decision is then added to the training data 1504 and the model is re-trained 1505. The rules include code matching, phonetic matching, distance matching, date matching, and/or partial string matching. FIG. 16 shows the ability for users to prepare for doctors' appointments by filling in a questionnaire and adding notes and questions that they want to discuss with their doctor. At the time of the appointment, a toggle will appear on the home screen to enable Doctor Mode. All information from the questionnaire, notes, and relevant information will then appear on the following screen.

The patient-centric Portal also has several functions providing the following ability:
Ability to input “notes” in both structured and non-structured format and search those notes.
Ability to proceed directly to a “satellite” portal from the Portal without additional authorization. The ability to differentiate between patient-entered information and provider-entered information.
Ability to access data directly from an EMR system for a specific patient with the ability to differentiate between the source of the data.
Track/audit applicable actions (manual and automatic) and provide reporting capability to meet current and future patient engagement and related ONC meaningful use measures. Ability to send the necessary information that confirms activity on behalf of the patient to satisfy the provider's meaningful use requirements relative to the CMS EHR Incentive Program for Stages 1, 2, and 3.
Ability to configure the user interface including level of data views, permissions granted to authorized representatives, and usability.
Ability to “hide” certain information based on the permission level granted.
Alerts generated from the “satellite” portal can be pushed to the patient's (or authorized representative) personal email, IM, and or mobile device in addition to alerts that are generated at the “core” Portal. All sources and time/date of alerts are identified and recorded.
Ability to generate real-time (24/7/365) and on demand drug-drug and drug-allergy alerts. Ability to configure clinical decision support rules and send alerts on a real-time basis to identified recipients. Automatic checking is performed upon addition of any new relevant clinical data.
Ability to generate, view, download and transmit a reconciled clinical summary (from multiple clinical summaries) in C-CDA, Blue Button, PDF, or ASCII text formats, among others.
Ability to configure the automatic sending of secure messages containing clinical data and/or system generated alerts received from one health care provider to one or more additional members of the patient's healthcare team.
Ability to generate search results via: the HL-7 InfoButton standard, a general search capability, and Google Alerts.
Ability to send and receive secure messages and produce related meaningful use audits and reports.
The ability to automatically present the patient's insurance eligibility in real-time including deductibles, co-pays, formularies. Ability to view/manage their Health Savings Account. The ability to monitor healthcare related spending (managing deductibles and/or co-pays throughout the year). This will include alerts that signify when deductibles have been met.
Ability to present a reference guide to current medications (with pill pictures).
Ability to maintain directory and contact information (including hyperlinks) for providers, care team members, home health care companies, pharmacies, and related entities.
Ability to trigger reminders to take appropriate medication.
Auto population of pharmacies, durable medical equipment supplies, hospitals, diagnostic facilities in the patient's geographic area (based on patient zip code on other location identifier, IP address, etc. Provided in list and/or map format.
Ability to provide cost information for treatment/diagnostic procedures, medical supplies, and patient medication based on patient zip code on other location identifier (IP address, etc.).
Ability to modify patient location manually to generate customized list and/or maps of healthcare related providers, suppliers, pharmacies, or resources
Ability to display trending data representation for lab results, exercise goals, nutritional intakes, and vital signs.
Ability to incorporate real-time audio/video feed to 2 or more nodes to be the access point for an on-line clinical encounter with providers, authorized representatives, and case managers.
Ability for users to consent to share their data with researchers.
Ability for users to get paid for contributing or sharing their data.
Ability to flag false information within a user's health records and provide that information back to the original source of the data.

Arranging for an Initial Medical Record by a User

A user has the ability to create her/his own combined medical record by sending a plurality of queries, called a User Initiated Super Query, to network endpoints that may contain a user's health data. With the present invention, a user can enter requisite information to request each of the various patient record keeping systems to provide the patient's data. The system of the present invention is configured to recognize and uniformly structure the received data to assist the de-duplication, diagnosis, and treatment determination processes.

A health care provider can also provide the patient's data directly to the system of the present invention. Using a health care provider portal, a health care provider can enter requisite information to be forwarded to the system of the present invention.

Portable Device Data

In addition to the data aggregated from queried endpoints, the system of the present invention allows users to link various devices that now are capable of transmitting real-time data in the home, either via Bluetooth, Wi-Fi or as yet to be developed near-field technologies for data entry, display, or access. Examples of this are glucometers that transmit blood-sugar levels after a test, and scales monitoring a patient weight. The system collects this data and presents it in an ongoing, chart format for the patient to see their results. The system triggers an alert when trends or results would, under circumstances either set by the user or polled from third-party sources, require special attention. This alert can be set to notify the healthcare professional as well as the patient.

Pharmaceutical Information

In addition to the cost-related data, the system allows a user to see an actual image of the medication they are taking, obtained from a third-party database. This medication identifier can be used in combination with a medication reminder application (displayed on, for example, smart phones and or television monitors) to assist the patient choose the correct medication when there are many to choose. The system also provides a user with any drug to drug and or drug to allergy interaction information, based upon the medications the particular user is taking. Standard, federally mandated patient disclosures about the medications presented, along with summary information available through third-party websites which display within a window on the display. An additional feature allows the user to elect to hear a synthesized or pre-recorded voice to read the key patient data for medications (e.g., side effects, contraindications, etc.), in a manner similar to what is often recited in television and radio advertisements for medications.

Data collected and included in the patient record of the present invention include (but are not limited to) patient problems, diagnoses, procedures, observations, family history, vaccinations, medications, and allergies. Data sources include health care providers, pharmacies, the patient, hospitals, insurance companies, medical and fitness devices, and potentially other family members. All records are kept confidential.

In one embodiment of the present invention, the system of the present invention learns on a patient by patient basis in order to determine items such as duplicity, diagnosis and treatment. As an example, a merged record might identify a variety of medical conditions, allergies, and medications related to a patient. The system of the present invention includes a populated (and potentially continually populated) rules database with rules for identifying allergies. In the example method of the present invention, when a patient's record adds a new medication, the rules database is implemented to determine if there is a potential for an allergic reaction between the newly entered medication and anything else in the patient record. This rules database includes both actually known allergy situations and “like” situations, and determines a confidence interval around a possible allergy. If the confidence interval is such that the potential allergy is inconclusive, a second rule is implemented, which could be comparison to similarly situated patients. The rules database of the present invention would then be updated based on an improved confidence interval.

In another example, matching rules are expandable by comparison, such as matching similarities in names, similarity in geographic location, similarity in dates, or similarity in partial strings. Rules can be established such that different confidence levels can be allotted for different types of similarities.

In the method of the present invention Data Aggregation, Machine Learning, and User in the Loop are operated together.

With regard to data aggregation, individual medical records are saved as files in a user's account. The medical record is then run through an Integration Engine. Data comes out of the Integration Engine.

One important attribute of the present invention is machine learning for de-duplication. Relative to Machine Learning: a de-duplication algorithm is used. Data is compared to contents of a user's current aggregate health record. Data is categorized into three “buckets”: same, possibly the same or possibly different, or different. If the data is exactly or sufficient (as defined in the algorithm) the same as what is already in the aggregate health record, it is not imported into the user's aggregate health record other than perhaps as indication of date of event (or some other attributes to show repeat occurrences). If the data is uniquely different from what is already in the aggregate health record, it is imported into the user's aggregate health record. If the algorithm cannot determine if the data is exactly the same or uniquely different, then the data are presented to the user.

User in the loop means bringing the user into the validation process of the data. If the algorithm cannot determine if the data is exactly the same or uniquely different, then the data will be presented to the user for the user to make the determination if the data is the same or different. Users can choose whether to add or not to add the data to their aggregate health record. The results of the user's selection ties back to the algorithm to tweak confidence levels and matching ranges.

While a number of examples and embodiments have been described, those of skill in the art will recognize certain modifications, additions or combinations that may be made to the specifics without deviating from the present inventive concepts. It is therefore intended that the invention be what is described in the following appended claims.

1. A method for a system comprising a processor, a data store for storing data records and processing rules, and a graphical user interface (GUI), and a communications interface, for formulating a concise and comprehensive patient medical record for determining potential patient medical conditions, comprising the steps of:
opening a GUI for a patient, said GUI remotely accessible by said patient;
upon identity verification of said patient, a processor communicating with a first and a subsequent remote medical record system and receiving a first and a subsequent set of medical data elements of said patient, where a data element is at least a portion of at least a notation in a patient's medical history;
said processor parsing said received first and subsequent sets of medical data elements for conformance to a data structure of a data store and storing said parsed data elements in said data store, thereby creating a comprehensive unfiltered patient medical record;
based on stored rules for elimination, said processor analyzing said comprehensive unfiltered patient medical record to identify and remove any determined redundant data elements, thereby forming and storing a comprehensive filtered medical record; and
based on stored rules for determining medical conditions, analyzing said filtered medical record to determine potential medical conditions of said patient.

2. The method of claim 1, wherein said rules for elimination include identifying similar or the same at least one of diagnoses, allergies, procedures, immunizations, vital signs, and other clinical data elements in different entries.

3. The method of claim 1, wherein said rules for elimination include determining redundant data elements based on calculated confidence intervals.

4. The method of claim 3, wherein, depending on the confidence interval, determined redundant data elements are either automatically eliminated from the filtered record, automatically retained in the filtered record, or presented to the patient for selection for addition or elimination.

5. The method of claim 4, wherein said elimination rules are adjusted based on patient selections.

6. The method of claim 1, wherein said rules for determining medical conditions are used to determine a plurality of data elements which collectively relate to a condition and said data elements are aggregated into a single record in said filtered patient medical records.

7. The method of claim 1, wherein said rules for determining medical conditions are used to determine a plurality of data elements which relate to a condition and said data elements are identified to the patient for selection for elimination of redundancy.

8. A method for a system comprising a processor, a data store for storing data records and processing rules, and a communication interface, to formulate an on-going concise and comprehensive patient medical record for determining patient adverse medical conditions, comprising the steps of:
upon identity verification of a patient, a processor communicating with a plurality of external medical record systems and retrieving multiple sets of data elements related to medical records of said patient;
said processor parsing said received sets of data elements for conformance to a data structure of a data store and storing said parsed data elements in said data store, thereby creating a comprehensive unfiltered patient medical record;
based on stored rules for elimination, said processor analyzing said comprehensive unfiltered patient medical record to identify and remove redundant data elements and, depending upon a determined confidence interface, offering at least one data element to a patient for determining redundancy and removing redundant data elements, thereby forming a filtered medical record; and
based on said stored rules for determining medical conditions, analyzing said filtered medical record to identity potentially interactive or adverse medications being administered to said patient and providing a notification of said situation to said patient.

9. The method of claim 8, further comprising the step of automatedly regularly updating said filtered record by polling said medical record systems for new data elements.

10. The method of claim 9, wherein said rules for elimination include elimination based on an algorithm determining similar or the same diagnoses, allergies, immunizations, or similar clinical data elements in different entries.

11. The method of claim 9, wherein said rules are used to determine a plurality of data elements which relate to a condition or diagnosis and said data elements are identified to the patient for selection for elimination of redundancy.

12. The method of claim 11, wherein determined redundant data elements are identified to the patient for selection for elimination based on a rule set, where the rule set is configured to make a determination under select medical conditions and offer the patient a selection based on other medical conditions.

13. The method of claim 12, wherein said rule set is updated based on patient selection.

14. The method of claim 11, wherein rules for determining potential medical conditions are used to determine a plurality of data elements which relate to a medical condition and said data elements are aggregated into a single record.

15. A system for formulating a concise and comprehensive patient medical record for identification of patient medical conditions comprising:
a processor for processing rules;
a data store for storing rules and patient medical histories;
a communication interface for communicating to medical record systems; and
a parsing engine for parsing records and determining redundancy;
wherein said system creates a comprehensive patient medical record by the processor, upon identity verification by a patient, receiving in response to requests for medical data elements for a patient from a plurality of external medical record systems; said processor parsing said received medical data elements for conformance to a data structure of a data store and storing said parsed medical records in said data store, thereby creating a comprehensive unfiltered patient medical record; based on stored rules for elimination, said processor analyzing said comprehensive unfiltered patient medical record to identify and remove redundant data elements thereby forming a filtered medical record; and based on said stored rules, analyzing said filtered medical record to identity potentially interactive or adverse medications being administered to said patient.

16. The system of claim 15, wherein said rules for elimination include elimination based on an algorithm determining similar or the same diagnoses in different entries.

17. The system of claim 15, wherein said rules for elimination include elimination based on an algorithm determining similar or the same prescriptions in different entries.

18. The system of claim 15, wherein determined redundant records are identified to the patient for selection for elimination.

19. The system of claim 15, wherein said rules are used to determine a plurality of data elements which relate to a condition and said data elements are aggregated into a single record.

20. The system of claim 15, wherein said rules are used to determine a plurality of data elements which relate to a condition and said data elements are identified to the patient for selection for elimination of redundancy.
